(19) United States
(12) Patent Application Publication
(10) Pub. No.: US 2026/0294332 A1
(43) Pub. Date: Oct. 1, 2026

(54) SYSTEM, METHOD AND NON-TRANSITORY COMPUTER-READABLE MEDIUM FOR SUPPORTING RECORDING OF INFORMATION REGARDING PRESSURE ULCER OF PATIENT

(71) Applicants: NEC Corporation, Tokyo (JP); JAPAN INSTITUTE FOR HEALTH SECURITY, Tokyo (JP)

(72) Inventors: Takafumi YOSHINAGA, Tokyo (JP); Shohei YAMAGUCHI, Tokyo (JP); Kengo MIYO, Tokyo (JP); Ryota NISHI, Tokyo (JP)

(73) Assignees: NEC Corporation, Tokyo (JP); JAPAN INSTITUTE FOR HEALTH SECURITY, Tokyo (JP)

(21) Appl. No.: 19/632,658

(22) Filed: Mar. 30, 2026

(30) Foreign Application Priority Data
Mar. 31, 2025 (JP) 2025-057733

(51) Int. Cl. A61B 5/00; A61B 90/00; G06T 7/00; G06T 7/60

(52) U.S. Cl. CPC A61B 5/445; A61B 5/742; A61B 5/7475; A61B 90/06; G06T 7/0012; G06T 7/60; A61B 2090/061; G06T 2207/10004; G06T 2207/20081; G06T 2207/30096

(57) ABSTRACT

A pressure ulcer recording support system comprises at least one memory configured to store instructions and at least one processor. The at least one processor is configured to execute the instructions to: generate a depth image indicating a pressure ulcer of a patient by using distance information regarding the pressure ulcer calculated by a distance measurement sensor included in a wearable terminal device; detect the pressure ulcer from the depth image; calculate depth information indicating a depth of the detected pressure ulcer; and register the calculated depth information.

### INCORPORATION BY REFERENCE

This application is based upon and claims the benefit of priority from Japanese patent application No. 2025-57733, filed on Mar. 31, 2025, the disclosure of which is incorporated herein in its entirety by reference.

### TECHNICAL FIELD

The present disclosure relates to techniques for supporting recording of information regarding a pressure ulcer of a patient.

### BACKGROUND ART

Conventionally, various technologies for estimating a size of a pressure ulcer in a medical site have been proposed. As an example of such a technology, WO 2019/230724 A1 discloses an image processing system that estimates an area of a pressure ulcer that is an affected region, calculates a size and an area of the estimated affected region, and outputs the calculated size and area of the affected region.

### SUMMARY

However, the image processing system disclosed in WO 2019/230724 A1 has a problem that a depth of the pressure ulcer cannot be recorded.

In view of the above-described problem, an example object of the present disclosure is to provide techniques for recording a depth of a pressure ulcer.

A pressure ulcer recording support system according to an example aspect of the present disclosure comprises:
at least one memory configured to store instructions; and
at least one processor configured to execute the instructions to:
generate a depth image indicating a pressure ulcer of a patient by using distance information regarding the pressure ulcer calculated by a distance measurement sensor included in a wearable terminal device;
detect the pressure ulcer from the depth image;
calculate depth information indicating a depth of the detected pressure ulcer; and
register the calculated depth information.

a
A wearable terminal device according to an example aspect of the present disclosure comprises:
at least one memory configured to store instructions; and
at least one processor configured to execute the instructions to:
generate a depth image indicating a pressure ulcer of a patient by using distance information regarding the pressure ulcer calculated by a distance measurement sensor included in a wearable terminal device;
detect the pressure ulcer from the depth image;
calculate depth information indicating a depth of the detected pressure ulcer; and
register the calculated depth information.

A computer implemented method according to an example aspect of the present disclosure comprises:
generating a depth image indicating a pressure ulcer of a patient by using distance information regarding the pressure ulcer calculated by a distance measurement sensor included in a wearable terminal device;
detecting the pressure ulcer from the depth image;
calculating depth information indicating a depth of the detected pressure ulcer; and
registering the calculated depth information.

A non-transitory computer-readable medium storing a program according to an example aspect of the present disclosure is provided. The program causes a computer to: generate a depth image indicating a pressure ulcer of a patient by using distance information regarding the pressure ulcer calculated by a distance measurement sensor included in a wearable terminal device;
detect the pressure ulcer from the depth image;
calculate depth information indicating a depth of the detected pressure ulcer; and
output the calculated depth information.

According to the present disclosure, it is possible to provide techniques for recording a depth of a pressure ulcer.

### BRIEF DESCRIPTION OF THE DRAWINGS

FIG. 1 is a diagram illustrating an example of a pressure ulcer recording support system according to the present disclosure;

FIG. 2 is a diagram illustrating an example of a hardware configuration of a wearable terminal device according to the present disclosure;

FIG. 3 is a diagram illustrating an example of a hardware configuration of an information processing device according to the present disclosure;

FIG. 4 is a diagram illustrating an example of a functional configuration of the wearable terminal device according to the present disclosure;

FIG. 5 is a diagram illustrating an example of a functional configuration of the information processing device according to the present disclosure;

FIG. 6 is a diagram illustrating an example of a transmission history table;

FIG. 7 is a diagram illustrating an example of a functional configuration of an electronic medical record management device according to the present disclosure;

FIG. 8 is a diagram illustrating an example of an electronic medical record information table;

FIG. 9 is a sequence diagram illustrating an example of processing executed in the pressure ulcer recording support system according to the present disclosure;

FIG. 10 is a diagram illustrating an example of a superimposed image of a pressure ulcer;

FIG. 11 is a diagram illustrating an example of an image displayed on a display of the wearable terminal device;

FIG. 12 is a diagram illustrating an example of extraction of patient information by a patient information extraction model; and

FIG. 13 is a diagram illustrating main components included in the pressure ulcer recording support system according to the present disclosure.

### EXAMPLE EMBODIMENT

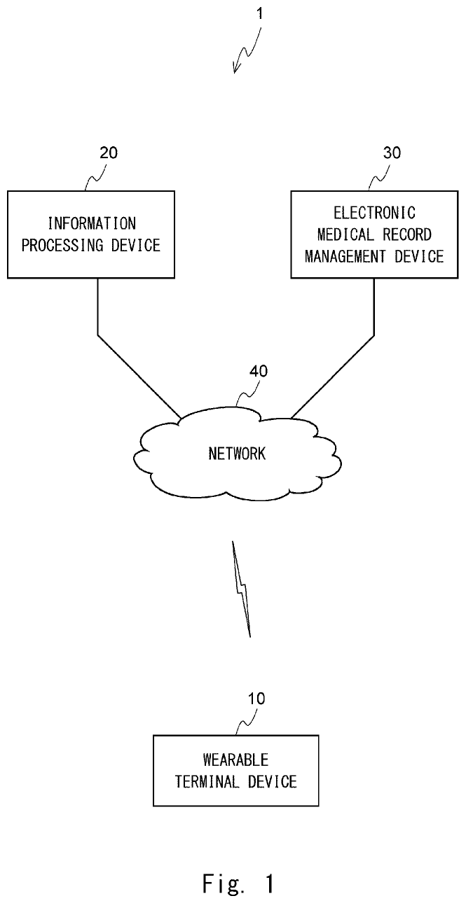

Hereinafter, an exemplary example embodiment will be described with reference to the drawings. FIG. 1 is a diagram illustrating an example of a pressure ulcer recording support system 1 according to the present disclosure. The pressure ulcer recording support system 1 includes a wearable terminal device 10, an information processing device 20, and an electronic medical record management device 30. Although FIG. 1 illustrates the single wearable terminal device 10, the pressure ulcer recording support system 1 may include a plurality of the wearable terminal devices 10.

The information processing device 20 communicates data with the wearable terminal device 10 and the electronic medical record management device 30 via a network 40. The network 40 may include a narrow area network such as a local area network (LAN) and/or a wide area network such as the Internet.

The wearable terminal device 10 is a terminal device worn by a medical worker. Specific examples of the wearable terminal device 10 include eyeglass-type wearable terminal devices such as smart glasses. The wearable terminal device 10 can transmit and receive data by wireless communication. The wearable terminal device 10 generates a captured image of a pressure ulcer of a patient, and provides the captured image to the information processing device 20. The wearable terminal device 10 acquires voice, and provides the voice to the information processing device 20. Details of a configuration of the wearable terminal device 10 will be described later.

The information processing device 20 calculates a depth and a size of a pressure ulcer by using a captured image of the pressure ulcer provided by the wearable terminal device 10. The information processing device 20 analyzes voice provided by the wearable terminal device 10, and generates text information regarding a patient. Specific examples of the information processing device 20 include an information processing device such as a server. Details of a configuration of the information processing device 20 will be described later.

The electronic medical record management device 30 is an information processing device that manages an electronic medical record in which information regarding a patient is registered. Specific examples of the electronic medical record management device 30 include an information processing device such as a server. The electronic medical record management device 30 registers the information regarding the patient provided by the information processing device 20 in an electronic medical record information table. Details of the electronic medical record information table will be described later.

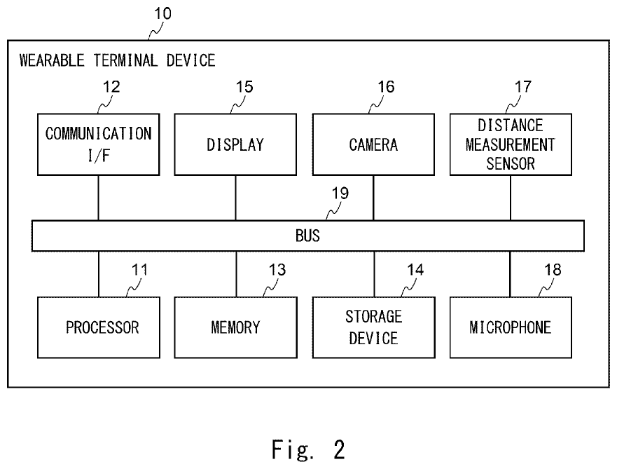

FIG. 2 is a diagram illustrating an example of a hardware configuration of the wearable terminal device 10. The wearable terminal device 10 includes a processor 11, a communication interface (I/F) 12, a memory 13, a storage device 14, a display 15, a camera 16, a distance measurement sensor 17, and a microphone 18. These devices exchange data via a bus 19.

The processor 11 is a device that performs overall control of the wearable terminal device 10. Specific examples of the processor 11 include a processor such as a central processing unit (CPU), a graphics processing unit (GPU), and a micro processing unit (MPU). The processor 11 loads a program stored in the storage device 14 into the memory 13 and executes the program, thereby executing a method defined by the program. In another example embodiment, a function executed by the processor 11 may also be executed by an integrated circuit such as a field-programmable gate array (FPGA) or an application specific integrated circuit (ASIC). These devices correspond to a computer.

The communication interface 12 is an interface that performs data communication between the wearable terminal device 10 and another device. The storage device 14 stores various types of data such as a program executed by the processor 11 and data processed by the processor 11.

The display 15 is a device that displays various images. Various types of information regarding a patient are displayed on the display 15. In a case where an eyeglass-type wearable terminal device is adopted as the wearable terminal device 10, the transmissive-type display 15 is disposed in front of eyes of a wearer of the wearable terminal device 10. The display 15 corresponds to a display unit.

The camera 16 is a device that captures an image under control of the processor 11. The camera 16 is preferably disposed in such a way as to be able to capture an image of a field of view of a wearer via the transmissive-type display 15.

The distance measurement sensor 17 is a device that measures a distance of an object under control of the processor 11. Specific examples of the distance measurement sensor 17 include a light detection and ranging (LiDAR) sensor. The distance measurement sensor 17 is preferably disposed in such a way as to be able to measure a region included in a field of view of a wearer.

The microphone 18 is a device for collecting voice of a wearer of the wearable terminal device 10. The microphone 18 corresponds to a sound collection unit.

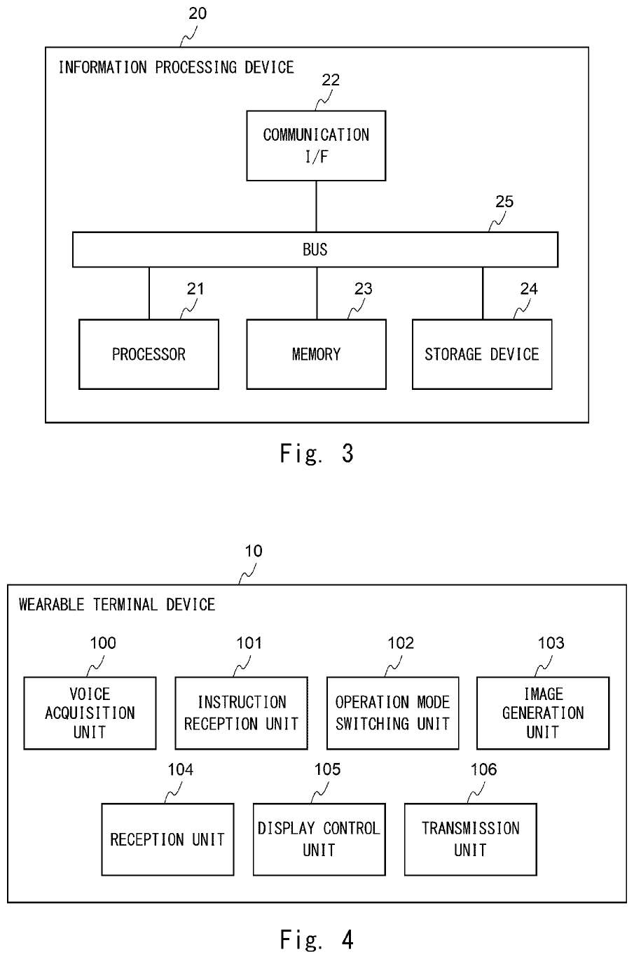

FIG. 3 is a diagram illustrating an example of a hardware configuration of the information processing device 20. The information processing device 20 includes a processor 21, a communication interface (I/F) 22, a memory 23, and a storage device 24. These devices exchange data via a bus 25. The electronic medical record management device 30 has the same hardware configuration as that of the information processing device 20.

The processor 21 is a device that performs overall control of the information processing device 20. Specific examples of the processor 21 include a processor such as a CPU, a GPU, and an MPU. The processor 21 loads a program stored in the storage device 24 into the memory 23 and executes the program, thereby executing a method defined by the program. In another example embodiment, a function executed by the processor 21 may also be executed by an integrated circuit such as an FPGA or an ASIC. These devices correspond to the computer.

The communication interface 22 is an interface that performs data communication between the information processing device 20 and another device. The storage device 24 stores various types of data such as a program executed by the processor 21 and data processed by the processor 21.

FIG. 4 is a diagram illustrating an example of a functional configuration of the wearable terminal device 10 according to the present disclosure. The wearable terminal device 10 includes a voice acquisition unit 100, an instruction reception unit 101, an operation mode switching unit 102, an image generation unit 103, a reception unit 104, a display control unit 105, and a transmission unit 106. These functional means can be achieved by a program.

The voice acquisition unit 100 acquires voice around the wearable terminal device 10, collected by the microphone 18.

The instruction reception unit 101 receives various instructions from a wearer. The instructions received by the instruction reception unit 101 include an instruction to switch an operation mode of the wearable terminal device 10, an instruction to capture an image of a pressure ulcer, and an instruction to register information regarding the pressure ulcer of a patient. These instructions can be given by the wearer by voice. These instructions can also be given by using an input device such as a button included in the wearable terminal device 10.

The operation mode switching unit 102 switches the operation mode of the wearable terminal device 10. The operation mode of the wearable terminal device 10 includes a normal operation mode and an automatic image capturing mode. In the automatic image capturing mode, the wearable terminal device 10 generates a two-dimensional captured image and a depth image.

The image generation unit 103 generates a two-dimensional captured image and a depth image of a pressure ulcer. Specifically, the image generation unit 103 causes the camera 16 to execute image capturing processing to generate the two-dimensional captured image of the pressure ulcer. The image generation unit 103 also causes the distance measurement sensor 17 to execute distance measurement processing to generate the depth image of the pressure ulcer from a measured value. In the automatic image capturing mode, the image generation unit 103 repeatedly causes the image capturing processing and the distance measurement processing to be executed to generate the two-dimensional captured images and the depth images of the pressure ulcer.

The reception unit 104 acquires various types of information from another device such as the information processing device 20. The information received by the reception unit 104 includes a superimposed image of a pressure ulcer, depth information regarding the pressure ulcer, size information regarding the pressure ulcer, and the like, which are generated by the information processing device 20.

The display control unit 105 displays various images on the display 15. In the images displayed on the display 15, a superimposed image of a pressure ulcer, depth information regarding the pressure ulcer, size information regarding the pressure ulcer, and the like, which are generated by the information processing device 20, are displayed.

The transmission unit 106 transmits various types of information to the information processing device 20. The information transmitted by the transmission unit 106 includes a two-dimensional captured image and a depth image generated by the image generation unit 103, distance information calculated by the distance measurement sensor 17, voice information acquired by the microphone 18, and the like.

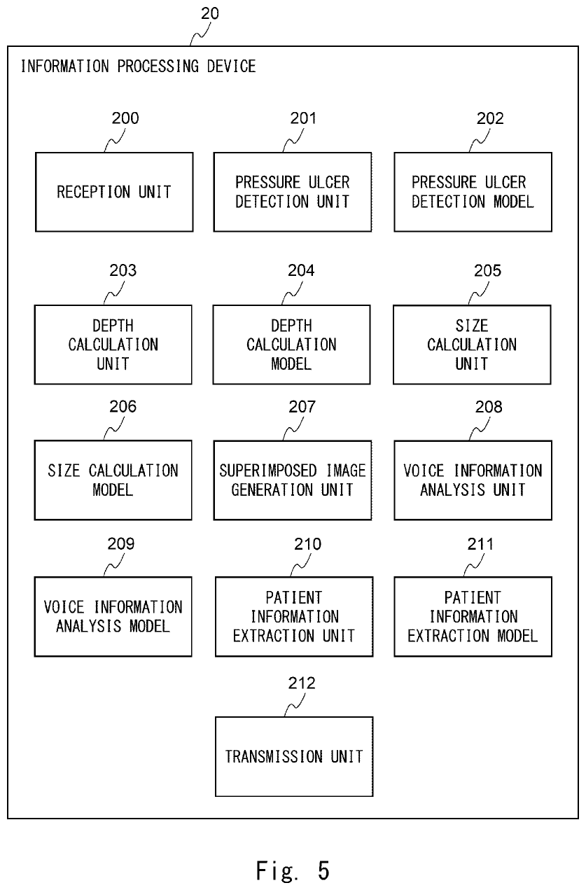

FIG. 5 is a diagram illustrating an example of a functional configuration of the information processing device 20 according to the present disclosure. The information processing device 20 includes a reception unit 200, a pressure ulcer detection unit 201, a pressure ulcer detection model 202, a depth calculation unit 203, a depth calculation model 204, a size calculation unit 205, a size calculation model 206, a superimposed image generation unit 207, a voice information analysis unit 208, a voice information analysis model 209, a patient information extraction unit 210, a patient information extraction model 211, and a transmission unit 212.

The reception unit 200 receives various types of information from another device. The information received by the reception unit 200 includes a two-dimensional captured image, a depth image, and measurement information, which are transmitted by the wearable terminal device 10.

The pressure ulcer detection unit 201 detects a pressure ulcer from a two-dimensional captured image and a depth image by using the pressure ulcer detection model 202.

The pressure ulcer detection model 202 is a machine learning model that detects, from a two-dimensional captured image and a depth image, a pressure ulcer included in these images. The pressure ulcer detection model 202 is trained to output, in a case where the two-dimensional captured image and the depth image each including the pressure ulcer are input, coordinate information that defines an image region of the pressure ulcer in each of these images. The pressure ulcer detection model 202 can be trained by machine learning such as deep learning.

The depth calculation unit 203 calculates, by using the depth calculation model 204, a depth of a pressure ulcer from a depth image in which the pressure ulcer is detected by the pressure ulcer detection unit 201.

The depth calculation model 204 is a machine learning model that calculates a depth of a pressure ulcer from a depth image in which the pressure ulcer is detected by the pressure ulcer detection unit 201. The depth calculation model 204 can be trained by machine learning such as deep learning. The depth calculation model 204 is trained to output, in a case where the depth image including the pressure ulcer and coordinate information that defines an image region of the pressure ulcer are input, depth information regarding the pressure ulcer included in the image region of the pressure ulcer. As the coordinate information that defines the image region of the pressure ulcer, for example, in a case where the image region of the pressure ulcer is displayed in a rectangular shape, coordinates of an end portion of the rectangular shape can be adopted.

The size calculation unit 205 calculates, by using the size calculation model 206, a size of a pressure ulcer from a two-dimensional captured image in which the pressure ulcer is detected by the pressure ulcer detection unit 201. The size of the pressure ulcer indicates, for example, a major axis and a minor axis of the pressure ulcer.

The size calculation model 206 is a machine learning model optimized to calculate a size of a pressure ulcer from a two-dimensional captured image in which the pressure ulcer is detected by the pressure ulcer detection unit 201. The size calculation model 206 can be trained by machine learning such as deep learning. The size calculation model 206 is trained to output, in a case where the two-dimensional captured image including the pressure ulcer, coordinate information that defines an image region of the pressure ulcer, a size of the image region of the pressure ulcer, and distance information provided by the wearable terminal device 10 are input, the size of the pressure ulcer included in the image region of the pressure ulcer. The size of the image region of the pressure ulcer is the size of the image region in which the pressure ulcer is displayed, and can be represented in pixels. The distance information provided by the wearable terminal device 10 represents a distance from the distance measurement sensor 17 to the pressure ulcer. The size calculation model 206 can calculate the size of the pressure ulcer included in the image region based on a correspondence relationship between the distance information and the size of the image region in which the pressure ulcer is displayed.

The superimposed image generation unit 207 generates a superimposed image 50 by superimposing predetermined image objects 52 and 53 on a two-dimensional captured image 51. FIG. 10 is a diagram illustrating an example of the superimposed image 50 of a pressure ulcer, generated by the superimposed image generation unit 207. The superimposed image 50 of the pressure ulcer includes the image object 52 indicating focus and the image object 53 indicating a scale. The superimposed image generation unit 207 can superimpose the image object 53 related to a size of the pressure ulcer calculated by the size calculation unit 205 on the two-dimensional captured image 51. The image object 53 indicating the scale may represent any length unit, such as millimeters, centimeters, or inches.

The voice information analysis unit 208 analyzes, by using the voice information analysis model 209, voice information provided from the wearable terminal device 10.

The voice information analysis model 209 is a machine learning model that analyzes voice information provided from the wearable terminal device 10 and outputs character information included in the voice information. In the present example embodiment, a trained model that can perform voice recognition can be adopted as the voice information analysis model 209. The voice information analysis model 209 can be subjected to machine learning by using training data in which voice information including various conversations of people is used as input information and character information representing the conversation in characters is used as output information.

The patient information extraction unit 210 extracts, by using the patient information extraction model 211, patient information that is information regarding a patient from character information output by the voice information analysis unit 208. The patient information includes information regarding a pressure ulcer of the patient.

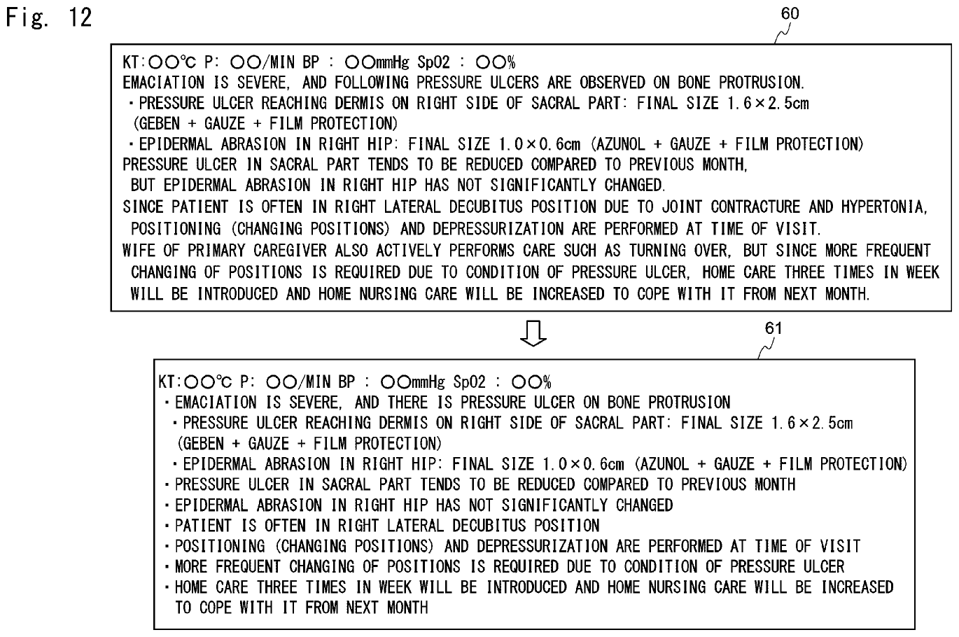

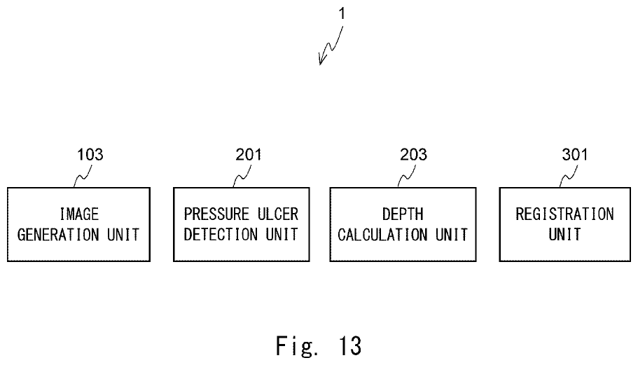

The patient information extraction model 211 is a machine learning model that extracts patient information from character information output by the voice information analysis model 209. In the present example embodiment, a language model such as large language models (LLM) can be adopted as the patient information extraction model 211. The patient information extraction model 211 is trained to output, in a case where the character information is input, text information that summarizes contents indicated by the character information. FIG. 12 is a diagram illustrating an example of the extraction of the patient information by the patient information extraction model 211. The patient information extraction model 211 can output, in a case where character information 60 is input, text information 61 that summarizes contents indicated by the character information. The text information 61 corresponds to the patient information.

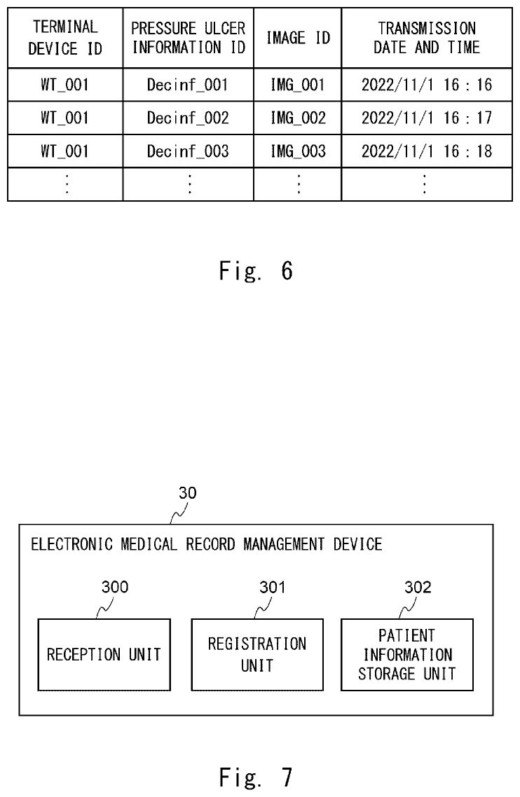

The transmission unit 212 transmits various types of information to another device. The information transmitted by the transmission unit 212 includes a superimposed image, depth information, size information, and identification information regarding pressure ulcer information, which are transmitted to the wearable terminal device 10. After transmitting these pieces of information to the wearable terminal device 10, the transmission unit 212 registers predetermined information in a transmission history table in which transmission history information is registered. FIG. 6 is a diagram illustrating an example of the transmission history table. In the transmission history table, identification information (terminal device identification (ID)) regarding the wearable terminal device 10 as a transmission destination of the superimposed image or the like, the identification information (pressure ulcer information ID) regarding the pressure ulcer information that is identification information regarding the transmitted depth information and size information, identification information (image ID) regarding the transmitted superimposed image, and a transmission date and time are registered in association with each other.

The information transmitted by the transmission unit 212 also includes patient information extracted by the patient information extraction unit 210 and transmitted to the electronic medical record management device 30. The transmission unit 212 transmits identification information (patient ID) regarding a patient together with the patient information extracted by the patient information extraction unit 210. The patient ID is provided by the wearable terminal device 10.

FIG. 7 is a diagram illustrating an example of a functional configuration of the electronic medical record management device 30 according to the present disclosure. The electronic medical record management device 30 includes a reception unit 300, a registration unit 301, and a patient information storage unit 302.

The reception unit 300 receives various types of information from another device. The information received by the reception unit 300 includes a patient ID, a superimposed image of a pressure ulcer, depth information regarding the pressure ulcer, size information regarding the pressure ulcer, and patient information, which are transmitted by the information processing device 20.

The registration unit 301 registers, in the electronic medical record information table, a superimposed image of a pressure ulcer, depth information regarding the pressure ulcer, size information regarding the pressure ulcer, and identification information regarding patient information, which are transmitted by the information processing device 20, in association with a patient ID.

The patient information storage unit 302 stores patient information transmitted by the information processing device 20 in a patient information database. The patient information database can be constructed in a storage device of the electronic medical record management device 30.

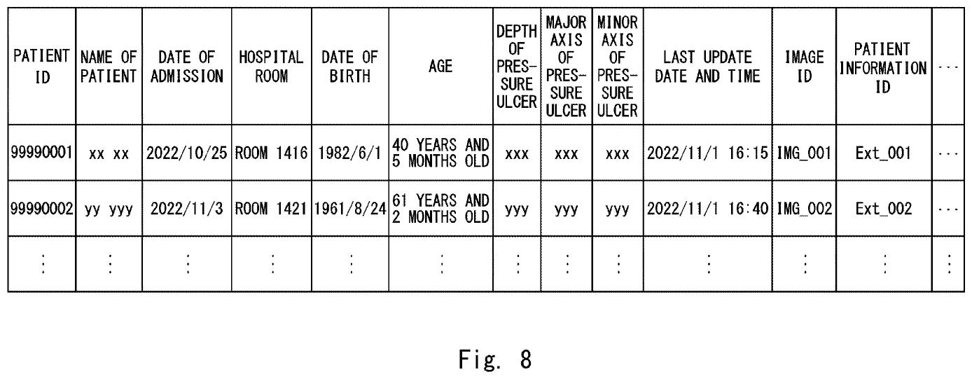

FIG. 8 is a diagram illustrating an example of the electronic medical record information table. In the electronic medical record information table, a patient ID, a name of a patient, a date of admission of the patient, a hospital room of the patient, a date of birth of the patient, an age of the patient, a depth of a pressure ulcer, a major axis and a minor axis of the pressure ulcer, a last update date and time, an image ID, a patient information ID, and the like are registered. The last update date and time is a last update date and time of the electronic medical record information table. The image ID is identification information regarding the superimposed image. The patient information ID is identification information regarding the patient information stored in the patient information database.

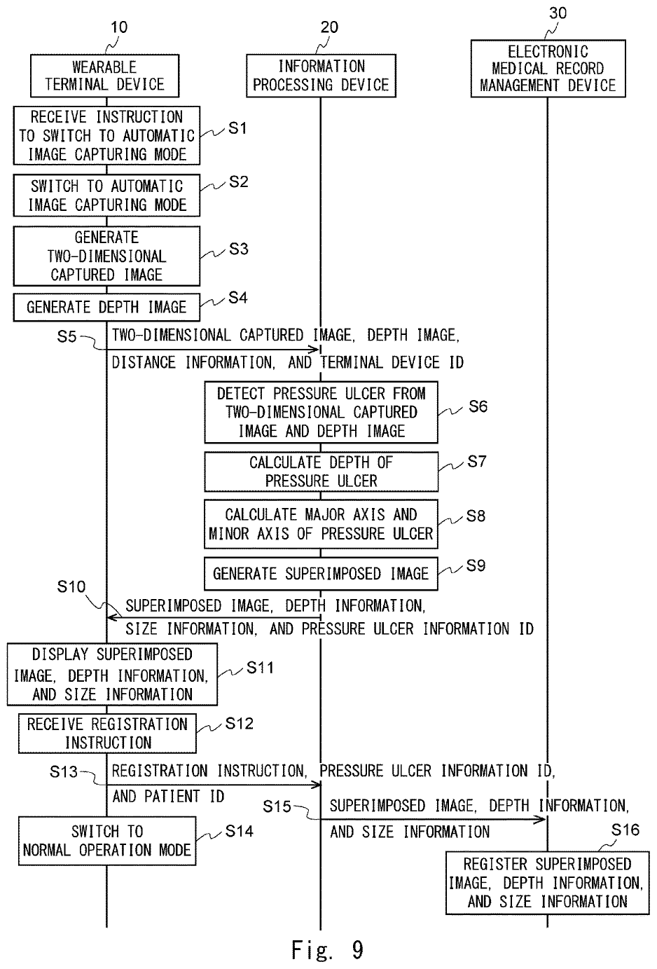

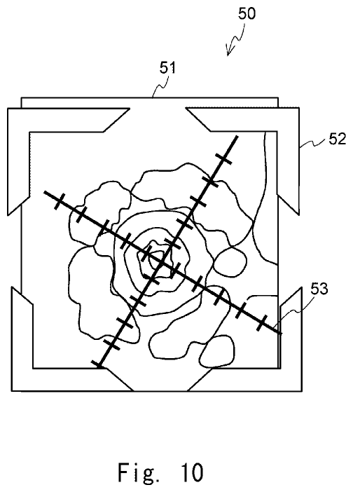

FIG. 9 is a sequence diagram illustrating an example of processing executed in the pressure ulcer recording support system 1. In step S1, the instruction reception unit 101 of the wearable terminal device 10 receives an instruction to switch to the automatic image capturing mode by a wearer of the wearable terminal device 10. In step S2, the operation mode switching unit 102 switches the operation mode of the wearable terminal device 10 to the automatic image capturing mode. In the automatic image capturing mode, the wearable terminal device 10 repeatedly executes processing of steps S3 to S5.

In step S3, the image generation unit 103 causes the camera 16 to execute the image capturing processing to generate a two-dimensional captured image. In step S4, the image generation unit 103 causes the distance measurement sensor 17 to execute the distance measurement processing to generate a depth image. In Step S5, the transmission unit 106 transmits, to the information processing device 20, the generated two-dimensional captured image and depth image and distance information calculated by the distance measurement sensor 17 together with identification information regarding the wearable terminal device 10.

After receiving the two-dimensional captured image, the depth image, the distance information, and the identification information regarding the wearable terminal device 10, which are transmitted by the wearable terminal device 10, the pressure ulcer detection unit 201 of the information processing device 20 detects a pressure ulcer from the two-dimensional captured image and the depth image in step S6. In step S7, the depth calculation unit 203 calculates a depth of the pressure ulcer. In step S8, the size calculation unit 205 calculates a major axis and a minor axis of the pressure ulcer. In step S9, the superimposed image generation unit 207 generates a superimposed image. In step S10, the transmission unit 212 transmits, to the wearable terminal device 10, the superimposed image, depth information, size information regarding the pressure ulcer, and identification information regarding pressure ulcer information. Here, the transmission unit 212 can specify the wearable terminal device 10 as a transmission destination by using the identification information regarding the wearable terminal device 10 received together with the two-dimensional captured image and the depth image.

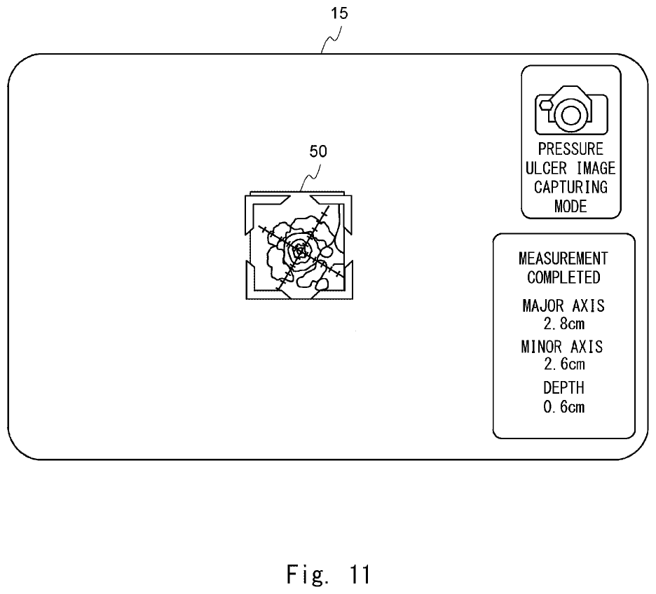

After receiving the superimposed image, the depth information, and the size information, which are transmitted by the information processing device 20, the display control unit 105 of the wearable terminal device 10 displays the received superimposed image, depth information, and size information on the display 15 in step S11. FIG. 11 is a diagram illustrating an example of an image displayed on the display 15 of the wearable terminal device 10. As illustrated in FIG. 11, the superimposed image 50, the depth information, and the size information are displayed on the display 15.

Next, after the wearer of the wearable terminal device 10 instructs the wearable terminal device 10 to register the information regarding the pressure ulcer, the instruction reception unit 101 of the wearable terminal device 10 receives the registration instruction of the information regarding the pressure ulcer in step S12. In step S13, the transmission unit 106 transmits the registration instruction, the pressure ulcer information ID, and a patient ID to the information processing device 20. The patient ID can be specified by the wearer of the wearable terminal device 10. In step S14, the operation mode switching unit 102 switches the operation mode of the wearable terminal device 10 to the normal operation mode.

After receiving the registration instruction, the pressure ulcer information ID, and the patient ID, which are transmitted by the wearable terminal device 10, the transmission unit 212 of the information processing device 20 transmits the superimposed image, the depth information, and the size information, which are associated with the pressure ulcer information ID in the transmission history table, to the electronic medical record management device 30 together with the patient ID in step S15.

After receiving the superimposed image, the depth information, the size information, and the patient ID, the registration unit 301 of the electronic medical record management device 30 stores the superimposed image, the depth information, and the size information in the electronic medical record information table in association with the patient ID in step S16.

FIG. 13 is a diagram illustrating main components included in the pressure ulcer recording support system 1. The pressure ulcer recording support system 1 includes the image generation unit 103, the pressure ulcer detection unit 201, the depth calculation unit 203, and the registration unit 301. The image generation unit 103 generates a depth image indicating a pressure ulcer of a patient by using distance information regarding the pressure ulcer calculated by the distance measurement sensor 17 included in the wearable terminal device 10. The pressure ulcer detection unit 201 detects the pressure ulcer from the depth image. The depth calculation unit 203 calculates depth information indicating a depth of the detected pressure ulcer. The registration unit 301 registers the calculated depth information. By adopting this configuration, the depth of the pressure ulcer can be recorded.

In the above-described example embodiment, the display control unit 105 displays the calculated depth information on the display 15 of the wearable terminal device 10. As a result, a wearer of the wearable terminal device 10 can confirm the depth information regarding the pressure ulcer.

In the above-described example embodiment, the instruction reception unit 101 receives an instruction from the wearer wearing the wearable terminal device 10. In a case where the instruction reception unit 101 receives an instruction to register information regarding the pressure ulcer displayed on the display 15 of the wearable terminal device 10, the registration unit 301 registers the depth information as electronic medical record information. By adopting this configuration, the wearer of the wearable terminal device 10 can instruct the registration of the depth information regarding the pressure ulcer at a timing at which the depth information regarding the desired pressure ulcer is displayed on the display 15, and the depth information can be registered as the electronic medical record information.

In the above-described example embodiment, the image generation unit 103 generates a captured image of the pressure ulcer by using the image capturing device 16 included in the wearable terminal device 10. In a case where the instruction reception unit 101 receives an instruction to register the information regarding the pressure ulcer displayed on the display 15, the registration unit 301 registers, as the electronic medical record information, the captured image of the pressure ulcer on which an image object indicating a scale is superimposed. By adopting this configuration, the captured image of the pressure ulcer on which the image object indicating the scale is superimposed can be registered as the electronic medical record information.

In the above-described example embodiment, the voice acquisition unit 100 acquires voice collected by the sound collection unit 18 included in the wearable terminal device 10. The voice information analysis unit 208 analyzes acquired voice information and outputs character information representing a word included in the voice information. The patient information extraction unit 210 extracts patient information regarding the patient from the output character information. The patient information storage unit 302 stores the extracted patient information in the storage device 14. By adopting this configuration, the wearer of the wearable terminal device 10 can register the patient information described orally.

In the above-described example embodiment, the wearable terminal device 10 automatically generates the two-dimensional captured image and the depth image of the pressure ulcer after shifting to the automatic image capturing mode, but in another example embodiment, the wearable terminal device 10 may generate the two-dimensional captured image and the depth image of the pressure ulcer each time in a case where an individual image capturing instruction by the wearer of the wearable terminal device 10 is received.

In the above-described example, the program includes instructions (or software code) for causing a computer to perform one or more functions described in the example embodiment in the case of being read by the computer. The program may be stored in a non-transitory computer readable medium or a tangible storage medium. As an example and not by way of limitation, the computer readable medium or the tangible storage medium includes a random-access memory (RAM), a read-only memory (ROM), a flash memory, a solid-state drive (SSD) or other memory technology, a CD-ROM, a digital versatile disk (DVD), a Blu-ray (registered trademark) disk, or other optical disk storages, a magnetic cassette, a magnetic tape, a magnetic disk storage, or other magnetic storage devices. The program may be transmitted on a transitory computer readable medium or a communication medium. As an example and not by way of limitation, the transitory computer readable medium or the communication medium includes electric, optical, acoustic, or other form of propagation signals.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with other embodiments.

Each of the drawings is merely illustrative for describing one or more example embodiments. Each of the drawings is not associated with only one specific example embodiment, but may be associated with one or more other example embodiments. As those of ordinary skill in the art will appreciate, various features or steps described with reference to any one of the drawings may be combined with features or steps illustrated in one or more other drawings, for example, to create an example embodiment that is not explicitly illustrated nor described. All of the features or steps illustrated in any one of the drawings for describing exemplary example embodiments are not necessarily mandatory, and some features or steps may be omitted. The order of the steps described in any of the drawings may be appropriately changed.

Some or all of the above example embodiments can also be described as the following Supplementary Notes, but are not limited to the following.

### Supplementary Note 1

A pressure ulcer recording support system comprising:
at least one memory configured to store instructions; and
at least one processor configured to execute the instructions to:
generate a depth image indicating a pressure ulcer of a patient by using distance information regarding the pressure ulcer calculated by a distance measurement sensor included in a wearable terminal device;
detect the pressure ulcer from the depth image;
calculate depth information indicating a depth of the detected pressure ulcer; and
register the calculated depth information.

### Supplementary Note 2

The pressure ulcer recording support system according to Supplementary Note 1, wherein the at least one processor is further configured to execute the instruction to display the calculated depth information on a display of the wearable terminal device to support decision making of a user wearing the wearable terminal device.

### Supplementary Note 3

The pressure ulcer recording support system according to Supplementary Note 2, wherein the at least one processor is further configured to execute the instructions to:
receive an instruction from a wearer wearing the wearable terminal device,
in a case of receiving an instruction to register information regarding the pressure ulcer displayed on the display, register the depth information as electronic medical record information.

### Supplementary Note 4

The pressure ulcer recording support system according to Supplementary Note 3, wherein the at least one processor is configured to:
generate a captured image of the pressure ulcer by using an image capturing device included in the wearable terminal device, and
in a case of receiving the instruction to register the information regarding the pressure ulcer displayed on the display, register, as the electronic medical record information, the captured image of the pressure ulcer on which an image object that indicates a scale is superimposed.

### Supplementary Note 5

The pressure ulcer recording support system according to Supplementary Note 1, wherein
the at least one processor is configured to calculate the depth information indicating the depth of the detected pressure ulcer by using a depth calculation model, and
the depth calculation model is a machine learning model optimized to output the depth information regarding the pressure ulcer in a case where the depth image and coordinate information that defines an image region of the pressure ulcer included in the depth image are input.

### Supplementary Note 6

The pressure ulcer recording support system according to Supplementary Note 1, wherein the at least one processor is further configured to execute the instructions to:
acquire voice collected by a sound collection unit included in the wearable terminal device;
analyze acquired voice information and outputs character information that represents a word included in the voice information;
extract patient information regarding the patient from the output character information; and
store the extracted patient information in a storage device.

### Supplementary Note 7

A wearable terminal device comprising:
a distance measurement sensor;
at least one memory configured to store instructions; and
at least one processor configured to execute the instructions to:
generate a depth image indicating a pressure ulcer of a patient by using distance information regarding the pressure ulcer calculated by the distance measurement sensor; detect the pressure ulcer from the depth image;
calculate depth information indicating a depth of the detected pressure ulcer; and
output the calculated depth information.

### Supplementary Note 8

The wearable terminal device according to Supplementary Note 7, wherein the at least one processor is further configured to execute the instruction to display the calculated depth information on a display of the wearable terminal device.

### Supplementary Note 9

The wearable terminal device according to Supplementary Note 8, wherein the at least one processor is further configured to execute the instructions to:
receive an instruction from a wearer wearing the wearable terminal device, and
in a case of receiving an instruction to register information regarding the pressure ulcer displayed on the display, register the depth information as electronic medical record information.

### Supplementary Note 10

The wearable terminal device according to Supplementary Note 9, wherein the at least one processor is configured to:
generate a captured image of the pressure ulcer by using an image capturing device included in the wearable terminal device, and
in a case of receiving the instruction to register the information regarding the pressure ulcer displayed on the display, register, as the electronic medical record information, the captured image of the pressure ulcer on which an image object that indicates a scale is superimposed.

### Supplementary Note 11

The wearable terminal device according to Supplementary Note 7 or 8, wherein
the at least one processor is configured to calculate the depth information indicating the depth of the detected pressure ulcer by using a depth calculation model, and
the depth calculation model is a machine learning model optimized to output the depth information regarding the pressure ulcer in a case where the depth image and coordinate information that defines an image region of the pressure ulcer included in the depth image are input.

### Supplementary Note 12

The wearable terminal device according to Supplementary Note 7 or 8, wherein the at least one processor is further configured to execute the instructions to:
acquire voice collected by a sound collection unit included in the wearable terminal device;
analyze acquired voice information and outputs character information that represents a word included in the voice information;
extract patient information regarding the patient from the output character information; and store the extracted patient information in a storage device.

### Supplementary Note 13

A computer implemented method comprising:
generating a depth image indicating a pressure ulcer of a patient by using distance information regarding the pressure ulcer calculated by a distance measurement sensor included in a wearable terminal device;
detecting the pressure ulcer from the depth image;
calculating depth information indicating a depth of the detected pressure ulcer; and registering the calculated depth information.

### Supplementary Note 14

A Program for causing a computer to:
generate a depth image indicating a pressure ulcer of a patient by using distance information regarding the pressure ulcer calculated by a distance measurement sensor included in a wearable terminal device;
detect the pressure ulcer from the depth image;
calculate depth information indicating a depth of the detected pressure ulcer; and
output the calculated depth information.

Some or all of the elements described in Supplementary Notes 2 to 6 depending from Supplementary Note 1 may depend from Supplementary Notes 13 and 14 as well with depending relationships similar to those of Supplementary Notes 2 to 6. Some or all of the elements described in any Supplementary Note may be applied to various types of hardware, software, recording means for recording software, systems, and methods.

1. A pressure ulcer recording support system comprising:
at least one memory configured to store instructions; and
at least one processor configured to execute the instructions to:
generate a depth image indicating a pressure ulcer of a patient by using distance information regarding the pressure ulcer calculated by a distance measurement sensor included in a wearable terminal device;
detect the pressure ulcer from the depth image;
calculate depth information indicating a depth of the detected pressure ulcer; and
register the calculated depth information.

2. The pressure ulcer recording support system according to claim 1, wherein the at least one processor is further configured to execute the instruction to display the calculated depth information on a display of the wearable terminal device to support decision making of a user wearing the wearable terminal device.

3. The pressure ulcer recording support system according to claim 2, wherein the at least one processor is further configured to execute the instructions to:
receive an instruction from a wearer wearing the wearable terminal device,
in a case of receiving an instruction to register information regarding the pressure ulcer displayed on the display, register the depth information as electronic medical record information.

4. The pressure ulcer recording support system according to claim 3, wherein
the at least one processor is configured to:
generate a captured image of the pressure ulcer by using an image capturing device included in the wearable terminal device, and
in a case of receiving the instruction to register the information regarding the pressure ulcer displayed on the display, register, as the electronic medical record information, the captured image of the pressure ulcer on which an image object that indicates a scale is superimposed.

5. The pressure ulcer recording support system according to claim 1, wherein
the at least one processor is configured to calculate the depth information indicating the depth of the detected pressure ulcer by using a depth calculation model, and
the depth calculation model is a machine learning model optimized to output the depth information regarding the pressure ulcer in a case where the depth image and coordinate information that defines an image region of the pressure ulcer included in the depth image are input.

6. The pressure ulcer recording support system according to claim 1, wherein the at least one processor is further configured to execute the instructions to:
acquire voice collected by a sound collection unit included in the wearable terminal device;
analyze acquired voice information and outputs character information that represents a word included in the voice information;
extract patient information regarding the patient from the output character information; and
store the extracted patient information in a storage device.

7. A computer implemented method comprising:
generating a depth image indicating a pressure ulcer of a patient by using distance information regarding the pressure ulcer calculated by a distance measurement sensor included in a wearable terminal device;
detecting the pressure ulcer from the depth image;
calculating depth information indicating a depth of the detected pressure ulcer; and
registering the calculated depth information.

8. The computer implemented method according to claim 7, wherein the method further comprises displaying the calculated depth information on a display of the wearable terminal device to support decision making of a user wearing the wearable terminal device.

9. The computer implemented method according to claim 8, wherein the method further comprises:
receiving an instruction from a wearer wearing the wearable terminal device, and
in a case of receiving an instruction to register information regarding the pressure ulcer displayed on the display, the registering comprises registering the depth information as electronic medical record information.

10. The computer implemented method according to claim 9, wherein the method further comprises generating a captured image of the pressure ulcer by using an image capturing device included in the wearable terminal device, and
in a case of receiving the instruction to register the information regarding the pressure ulcer displayed on the display, the registering comprises registering, as the electronic medical record information, the captured image of the pressure ulcer on which an image object that indicates a scale is superimposed.

11. The computer implemented method according to claim 7, wherein the method further comprises calculating the depth information indicating the depth of the detected pressure ulcer by using a depth calculation model, and
the depth calculation model is a machine learning model optimized to output the depth information regarding the pressure ulcer in a case where the depth image and coordinate information that defines an image region of the pressure ulcer included in the depth image are input.

12. The computer implemented method according to claim 7, wherein the method further comprises:
acquiring voice collected by a sound collection unit included in the wearable terminal device;
analyzing acquired voice information and outputs character information that represents a word included in the voice information;
extracting patient information regarding the patient from the output character information; and
storing the extracted patient information in a storage device.

13. A non-transitory computer-readable medium storing a program for causing a computer to:
generate a depth image indicating a pressure ulcer of a patient by using distance information regarding the pressure ulcer calculated by a distance measurement sensor included in a wearable terminal device;
detect the pressure ulcer from the depth image;
calculate depth information indicating a depth of the detected pressure ulcer; and
output the calculated depth information.
